(19) United States
(12) Patent Application Publication
(10) Pub. No.: US 2026/0094701 A1
(43) Pub. Date: Apr. 2, 2026

(54) MEDICAL SERVICE MANAGEMENT SYSTEM

(71) Applicant: Konica Minolta, Inc., Tokyo (JP)

(72) Inventor: Akinori TADOKORO, Tokyo (JP)

(21) Appl. No.: 19/340,305

(22) Filed: Sep. 25, 2025

(30) Foreign Application Priority Data
Sep. 30, 2024 (JP) 2024-171104

(51) Int. Cl. G16H 40/20; G16H 10/40; G16H 10/60; G16H 50/20; G16H 80/00

(52) U.S. Cl. CPC G16H 40/20; G16H 10/40; G16H 10/60; G16H 50/20; G16H 80/00

(57) ABSTRACT

An object is to provide a medical service management system capable of accurately managing the use of a medical service by SaMD. A medical service management system of the present disclosure is a medical service management system that manages use of a medical service by Software as a Medical Service (SaMD), in which the medical service management system causes: a first terminal in which an application of the medical service is installed, to output display information indicating a test result by the medical service; a second terminal linkable with an electronic medical record, to read the output display information; and the test result to be registered in the electronic medical record based on a result of the reading.

### CROSS REFERENCE TO RELATED APPLICATIONS

The entire disclosure of Japanese Patent Application No. 2024-171104 filed on Sep. 30, 2024, is incorporated herein by reference in its entirety.

### BACKGROUND

### Technological Field

The present disclosure relates to a medical service management system that manages use of a medical service.

### Description of Related Art

A medical system has been developed that provides an application (hereinafter, referred to as app) for processing output data from a medical apparatus such as an X-ray CT apparatus or a magnetic resonance diagnostic apparatus. Such a medical system uses a usage-based billing system.

As a technique for accurately charging a fee, Japanese Unexamined Patent Publication No. 2016-224676 discloses that the diagnostic effectiveness of an app used is determined and a fee is charged according to the effectiveness.

### SUMMARY

In recent years, the use of medical services using Software as a Medical Device (SaMD) has spread in clinical settings. SaMD is software having a complicated medical function. A doctor can use various medical services provided by SaMD on a terminal device by accessing the SaMD service in the cloud using a general-purpose terminal device, such as a tablet or a smartphone. By SaMD, various data obtained by various tests can be rapidly and accurately processed, and various medical practices such as diagnosis and treatment can be promoted. In addition, by SaMD, since SaMD can be used in a relatively small portable computer such as a tablet or a smartphone, medical practice can be performed anywhere.

In order to improve usability in clinical settings, it is desirable that a terminal device for using SaMD can be operated by an unspecified large number of persons. Examples of the unspecified large number of persons include nurses and patients in addition to doctors.

In order to reduce the risk of leakage of patient information or the like that is confidential information, it is desirable to make such a terminal device that can be operated by an unspecified large number of persons independent from an electronic medical record system that handles patient information.

However, when the terminal device for using SaMD and the electronic medical record system are made independent of each other in this way, the following disadvantages may occur. That is, it is difficult to automatically reflect the medical information acquired by the SaMD service in the electronic medical record in the terminal device. Therefore, a doctor is required to manually reflect the medical information acquired by the SaMD service in the electronic medical record, which increases a burden on the doctor.

Furthermore, the information on the usage history of the SaMD service is managed on the side of the server that provides the SaMD service and on the side of the electronic medical record system, respectively. Here, since the server and the electronic medical record system are independent of each other, a situation may occur in which the usage history of the SaMD service does not match between the server side and the electronic medical record system side. In such a case, it is difficult to calculate a correct usage fee for the SaMD service.

An object of the present disclosure is to provide a medical service management system capable of accurately managing the use of a medical service by SaMD.

To achieve at least one of the abovementioned objects, according to an aspect of the present invention, a medical service management system according to an aspect of the present disclosure is a medical service management system that manages use of a medical service by Software as a Medical Service (SaMD). The medical service management system causes: a first terminal in which an application of the medical service is installed, to output display information indicating a test result by the medical service; a second terminal linkable with an electronic medical record, to read the output display information; and the test result to be registered in the electronic medical record based on a result of the reading.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating an example of a configuration of a medical service management system according to Embodiment 1 of the present disclosure;

FIG. 2 is a block diagram illustrating an example of a functional configuration of an SaMD terminal device according to Embodiment 1;

FIG. 3 is a block diagram illustrating an example of a functional configuration of the electronic medical record terminal device according to Embodiment 1;

FIG. 4 is a sequence diagram illustrating an operation example of the medical service management system according to Embodiment 1;

FIG. 5 is a diagram illustrating an example of a configuration of the medical service management system according to Embodiment 2 of the present disclosure;

FIG. 6 is a block diagram illustrating an example of a functional configuration of a comparison server apparatus according to Embodiment 2; and

FIG. 7 is a flowchart illustrating an operation example in the comparison process of the comparison server apparatus according to Embodiment 2.

### DETAILED DESCRIPTION OF EMBODIMENTS

Hereinafter, one or more embodiments of the present invention will be described with reference to the drawings. However, the scope of the invention is not limited to the disclosed embodiments.

### Embodiment 1

Hereinafter, Embodiment 1 of the present disclosure will be described.

### <Overall Configuration>

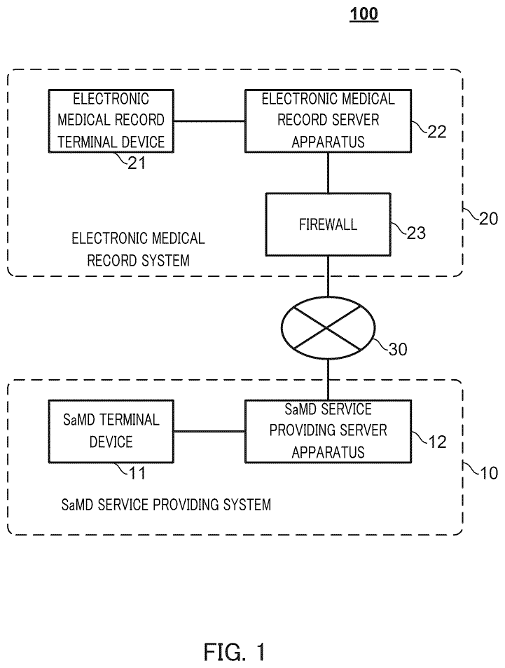

FIG. 1 is a diagram illustrating an example of a configuration of a medical service management system 100 according to Embodiment 1 of the present disclosure. In Embodiment 1, the medical service management system 100 includes a SaMD service providing system 10 and an electronic medical record system 20.

The SaMD service providing system 10 and the electronic medical record system 20 are connected to each other via a network (e.g., the internet) 30.

The SaMD service providing system 10 is a system that provides various types of medical services using SaMD to medical professionals such as doctors. The SaMD service providing system 10 is introduced to, for example, a medical institution such as a hospital.

In the present disclosure, a service of outputting a test result performed on a patient is assumed as a medical service by SaMD. A specific example of the medical service provided by SaMD according to the present disclosure includes a dementia test service in which a test of whether the patient suffers from dementia is conducted based on a patient's voice or the like, and the result of the test is provided. A doctor can appropriately make a diagnosis of a patient by utilizing a test result provided by a medical service based on SaMD.

The SaMD service providing system 10 includes a SaMD terminal device 11 and a SaMD service providing server apparatus 12.

The SaMD terminal device 11 is a terminal device for a medical professional such as a doctor to use a medical service by SaMD. In the present disclosure, examples of the SaMD terminal device 11 include portable computers such as tablet computers, smartphones, and notebook personal computers (PCs), and stationary computers such as desktop PCs.

The SaMD service providing server apparatus 12 is a server apparatus that provides a medical service by SaMD to the SaMD terminal device 11. Examples of the SaMD service providing server apparatus 12 include, for example, a physical server configured by a stationary computer, or a cloud server configured by deploying one or a plurality of virtualized server functions in a cloud environment.

The SaMD terminal device 11 and the SaMD service providing server apparatus 12 are connected to be communicable with each other. When a medical professional such as a doctor performs an operation for using a medical service by SaMD via the SaMD terminal device 11, the SaMD terminal device 11 requests the SaMD service providing server apparatus 12 to provide a service. When the SaMD service providing server apparatus 12 transmits various kinds of information for providing services to the SaMD terminal device 11, the medical professional can use the medical service by SaMD on the SaMD terminal device 11.

The electronic medical record system 20 is a system that electronically and centrally manages, as an electronic medical record, various kinds of information generated when a doctor examines a patient. Examples of various kinds of information generated when a doctor examines a patient include personal information on the patient, the result of a test performed on the patient, a test image, a test result by the doctor based on the test result or the test, an order of prescription, and accounting information. The electronic medical record system 20 is introduced to, for example, a medical institution such as a hospital in the same manner as the SaMD service providing system 10.

The electronic medical record system 20 includes an electronic medical record terminal device 21, an electronic medical record server apparatus 22, and a firewall 23. The electronic medical record server apparatus 22 is a server device that manages electronic medical records of a plurality of patients. The electronic medical record terminal device 21 is a terminal device that can access the electronic medical record server apparatus 22. In the present disclosure, examples of the electronic medical record terminal device 21 include portable computers such as tablet computers, smartphones, and notebook PCs, and stationary computers such as desktop computers. Furthermore, examples of the electronic medical record server apparatus 22 include, for example, a physical server composed of a stationary computer, or a cloud server configured by deploying one or a plurality of virtualized server functions in a cloud environment. When a doctor inputs a result of diagnosing a patient (hereinafter, referred to as a test result) via the electronic medical record terminal device 21, the test result is reflected in the electronic medical record managed by the electronic medical record server apparatus 22.

The firewall 23 restricts communication connection within and to the outside of the electronic medical record system 20. It is possible to reduce a risk that confidential information (e.g., personal information of a patient or information on a test result) handled within the electronic medical record system 20 leaks to the outside of the electronic medical record system 20. In particular, in the example illustrated in FIG. 1, the firewall 23 restricts communication between the SaMD service providing system 10 and the electronic medical record system 20.

### <SaMD Terminal Device 11>

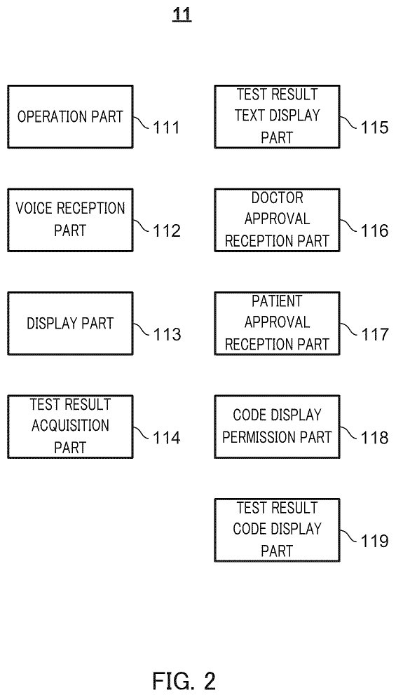

FIG. 2 is a block diagram illustrating an example of a functional configuration of the SaMD terminal device 11 according to Embodiment 1. As illustrated in FIG. 2, the SaMD terminal device 11 includes an operation part 111, a voice reception part 112, a display part 113, a test result acquisition part 114, a test result text display part 115, a doctor approval reception part 116, a patient approval reception part 117, a code display permission part 118, and an test result code display part 119.

The operation part 111 receives an operation of a person who uses the SaMD terminal device 11. The persons who use the SaMD terminal device 11 include at least a doctor. Furthermore, persons who use the SaMD terminal device 11 may include a nurse or various kinds of technicians who are medical professionals other than doctors, or a patient himself/herself. The operation part 111 includes, for example, various buttons, keys, switches, and a touch pad.

The voice reception part 112 receives the voice of the patient. The voice reception part 112 includes, for example, a microphone. The patient's voice received by the voice reception part 112 is transmitted to the SaMD service providing server apparatus 12.

The display part 113 displays various kinds of information handled in the SaMD terminal device 11. The display part 113 is a display device such as a liquid crystal display or an organic electro luminescence (EL) display.

In a case where the operation part 111 is composed of a touch pad, a so-called touch screen may be configured by the operation part 111 being superimposed on the display part 113.

The test result acquisition part 114 acquires a test result generated by the SaMD service providing server apparatus 12 on the basis of the voice of the patient. When the medical service provided by the SaMD service providing system 10 is a dementia test service, the test result acquired by the test result acquisition part 114 is a test result indicating whether or not the patient has dementia.

The test result text display part 115 displays, on display part 113, the test result acquired by the test result acquisition part 114 in a text format. Thus, the person who uses the SaMD terminal device 11 can accurately grasp the test result.

The doctor approval reception part 116 receives doctor's approval for the test result acquired by the test result acquisition part 114. When the doctor performs an operation for approval via the operation part 111, the doctor approval reception part 116 may receive the doctor's approval. Note that the approval by the doctor for the test result of a certain patient means that the doctor approves that the test result may be reflected in the electronic medical record of the patient.

The patient approval reception part 117 receives approval from the patient for the test result acquired by the test result acquisition part 114. When the patient performs an operation for approval via the operation part 111, the doctor approval reception part 116 may receive the approval from the patient. Note that the approval from a certain patient for the test result of the certain patient means that the patient is satisfied with the test result.

The test result code display part 119 causes the display part 113 to display the test result acquired by the test result acquisition part 114 in a barcode format. In the present disclosure, the barcode includes at least a one-dimensional barcode and a two-dimensional code. Examples of a two-dimensional code include a quick response (QR) code (registered trademark of DENSO WAVE INCORPORATED).

The test result in a barcode format which the test result code display part 119 causes the display part 113 to display is read by the electronic medical record terminal device 21. Thus, the test result acquired by the medical service provided by the SaMD service providing system 10 is automatically and accurately received by the electronic medical record system 20.

In the display part 113, the test result in a text format displayed by the test result text display part 115 and the test result in a barcode format displayed by the test result code display part 119 may be displayed at the same time in a state of being arranged side by side. Alternatively, in the display part 113, after the display of the test result in a text format by the test result text display part 115, the display of the test result in a barcode format by the test result code display part 119 may be performed.

The code display permission part 118 permits the display of the test result in a barcode format by the test result code display part 119 on condition that the doctor's approval and the patient's approval are received by the doctor approval reception part 116 and the patient approval reception part 117, respectively.

### <Electronic Medical Record Terminal Device 21>

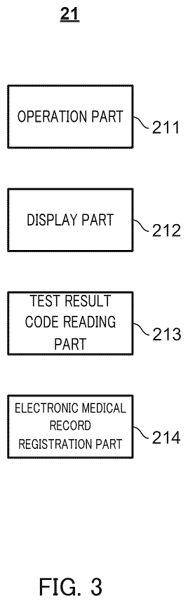

FIG. 3 is a block diagram illustrating an example of a functional configuration of the electronic medical record terminal device 21 according to Embodiment 1. As illustrated in FIG. 3, the electronic medical record terminal device 21 includes an operation part 211, a display part 212, a test result code reading part 213, and an electronic medical record registration part 214.

The operation part 211 receives an operation of a doctor who uses the electronic medical record terminal device 21. The operation part 211 includes, for example, various buttons, keys, switches, and a touch pad.

The display part 212 displays various kinds of information handled in the electronic medical record terminal device 21. The display part 212 is a display device such as a liquid crystal display or an organic EL display, for example. In a case where the operation part 211 is composed of a touch pad, a so-called touch screen may be configured by the operation part 211 being superimposed on the display part 212.

The test result code reading part 213 reads the test result in a barcode format displayed on the display part 113 by the test result code display part 119 of the SaMD terminal device 11. The test result code reading part 213 has, for example, a barcode reader function, and reads a barcode displayed on the display part 113 by binarizing the barcode.

The electronic medical record registration part 214 transmits the contents of the test result read by the test result code reading part 213 to the electronic medical record server apparatus 22, thereby registering the test result in the electronic medical record managed by the electronic medical record server apparatus 22. In addition to the above-described process, the electronic medical record registration part 214 may perform a process of registering information to be registered in the electronic medical record, which is input by the doctor, in the electronic medical record by transmitting the information to the electronic medical record server apparatus 22. Examples of information to be registered in the electronic medical record include patient information and diagnostic information.

### <Operation Example of Medical Service Management System 100>

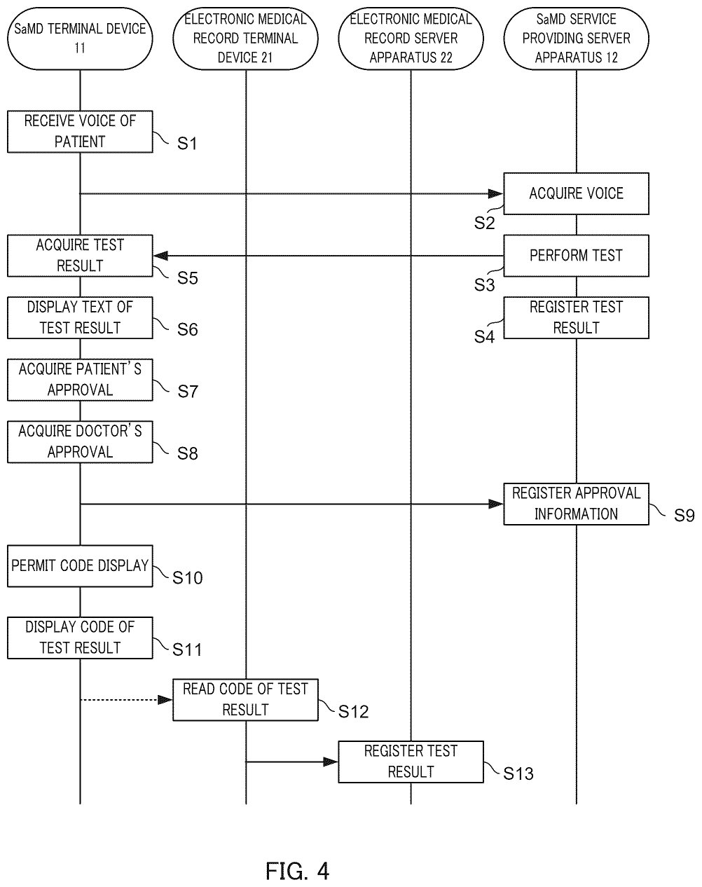

An operation example of the medical service management system 100 according to Embodiment 1 will be described with reference to FIG. 4. FIG. 4 is a sequence diagram illustrating an operation example of the medical service management system 100 according to Embodiment 1. An arrow between devices illustrated in FIG. 4 means that some information is exchanged.

In the following description, as a medical service provided by the SaMD service providing system 10, a dementia test service by SaMD is assumed. The dementia test service is a service for conducting a test whether the patient has dementia based on the voice of a patient and providing a test result thereof.

A medical professional such as a doctor or a nurse operates the SaMD service providing system 10 to start using the dementia test service by SaMD. In Step S1, the SaMD terminal device 11 receives the patient's voice used for the test of the dementia test service by the operation of the medical professional or the patient himself/herself. The voice of the patient received by the SaMD terminal device 11 in Step S1 is transmitted to the SaMD service providing server apparatus 12.

In Step S2, the SaMD service providing server apparatus 12 acquires the voice of the patient transmitted from the SaMD terminal device 11.

In Step S3, the SaMD service providing server apparatus 12 performs a test to determine whether or not the patient has dementia by using the voice of the patient acquired in Step S2. The test may be performed, for example, using a trained model that outputs an index value indicating the degree of dementia of a patient when the patient's voice is input. In this case, the test result is the index value itself or the presence or absence of dementia determined based on the index value.

In Step S4, the SaMD service providing server apparatus 12 registers the test result acquired by the test performed in Step S3. Note that registering a test result means that the SaMD service providing server apparatus 12 does not store information on the patient on which the test has been performed, and stores only information on the date and time when the test has been performed and information indicating the contents of the test result. Examples of the information on the patient on which the test has been performed include the patient's name, gender, age, and address. As a result, information on the patient, which is confidential information, is not stored in the SaMD service providing server apparatus 12. Therefore, it is possible to reduce the situation in which information regarding the patient, which is confidential information, leaks to the outside via the SaMD service providing server apparatus 12 due to use of the SaMD service.

In Step S5, the SaMD terminal device 11 acquires the test result transmitted from the SaMD service providing server apparatus 12.

In Step S6, the SaMD terminal device 11 displays the test result acquired in Step S5 in a text format. Thus, the person using the SaMD terminal device 11 can grasp the test result of the patient.

In Step S7, the SaMD terminal device 11 acquires the patient's approval. The patient's approval means that the patient receives the contents of the test result displayed in Step S6. In a case where the patient's approval is not acquired in Step S7, the process of Step S7 may be repeatedly executed until the patient's approval is acquired, or the process may return to Step S1 and the test may be performed again from the process of receiving the patient's voice.

In Step S8, the SaMD terminal device 11 acquires the doctor's approval. The doctor's approval means that the doctor approves that the test result displayed in Step S6 is a formal test result that canbe registered in the electronic medical record. When the doctor's approval is not acquired in Step S8, the process of Step S8 may be repeatedly executed until the doctor's approval is obtained, or the process may return to Step S1 and the test may be performed again from the process of receiving the voice of the patient. The SaMD terminal device 11 that has acquired the doctor's approval in Step S8 transmits approval information indicating that the doctor's approval has been acquired, to the SaMD service providing server apparatus 12.

In Step S9, the SaMD service providing server apparatus 12 registers the approval information transmitted from the SaMD terminal device 11 in association with the test result registered in Step S4. This makes it clear that the test result registered in the SaMD service providing server apparatus 12 has been approved by the doctor.

In Step S10, in response to acquiring the doctor's approval in Step S8, the SaMD terminal device 11 permits display of the test result acquired in Step S5 in a barcode format.

In Step S11, in response to the permission in Step S10, the SaMD terminal device 11 displays the test result acquired in Step S5 in a barcode format. As described above, the test result in a text format displayed in Step S6 and the test result in a barcode format displayed in Step S11 may be displayed at the same time in the same display part side by side with each other. Alternatively, after the display of the test result in a text format is performed in Step S6, the display of the test result in a text format may be stopped, and then the display of the test result in the barcode format may be performed in Step S11.

In Step S12, the electronic medical record terminal device 21 reads the test result code displayed in Step S9. Since the reading of the test result code in Step S12 is not realized by transmitting any information from the SaMD terminal device 11 to the electronic medical record terminal device 21, the reading of the test result code is indicated by a broken line arrow in FIG. 4. The electronic medical record terminal device 21 transmits the test result acquired from the test result code read in Step S12 to the electronic medical record server apparatus 22.

In Step S13, the electronic medical record server apparatus 22 registers the test result transmitted from the electronic medical record terminal device 21 in the electronic medical record. Thus, the test result acquired by using the medical service by SaMD is automatically registered in the electronic medical record. Thus, when the test result obtained by using the medical service by the SaMD is registered in the electronic medical record, for example, it is not necessary for the doctor to spend much labor such as operating the electronic medical record terminal device 21 based on the test result displayed in a text format on the SaMD terminal device 11.

### Effect

Effects obtained by the medical service management system 100 will be described in detail on the basis of the operation example of the medical service management system 100 illustrated in FIG. 4.

As described above, the SaMD terminal device 11 for using the SaMD can be used by unspecified persons such as medical professionals other than doctors and patients themselves. Accordingly, it is possible to flexibly use the medical service (the dementia test service in the example of FIG. 4) provided to the SaMD terminal device 11 by the SaMD service providing server apparatus 12 depending on the situation in clinical settings. For example, a doctor or a nurse can use the SaMD terminal device 11 to provide a patient with an explanation about a medical service that the patient will use. Then, the acquisition of the voice of the patient by the SaMD terminal device 11, which is necessary for the execution of the medical service, can be performed by the patient himself/herself operating the SaMD terminal device 11 or can be performed by a doctor or a nurse operating the SaMD terminal device 11.

Meanwhile, the communication with the electronic medical record server apparatus 22 is restricted by the firewall 23 of the electronic medical record system 20 in such a SaMD service providing system 10 including the SaMD terminal device 11 usable by unspecified persons. Accordingly, it is possible to suppress a risk that confidential information such as personal information of a patient or a test result, which is handled in the electronic medical record system 20, leaks to the outside via the SaMD terminal device 11.

Then, as shown in Step S6 of FIG. 4, the SaMD terminal device 11 displays the test result acquired from the SaMD service providing server apparatus 12 in a text format. Thus, the person using the SaMD terminal device 11 can grasp the contents of the test result. Therefore, in Step S7 and Step S8, the patient and the doctor can sufficiently understand the contents of the test result and then approve the contents of the test result.

In addition, as illustrated in Step S9 of FIG. 4, the SaMD terminal device 11 displays, in a barcode format, the test result acquired from the SaMD service providing server apparatus 12 after the approval of the patient and the doctor in Steps S7 and S8. Then, the electronic medical record terminal device 21 reads the test result displayed in a barcode format by the barcode reader function. Thus, the test result can be accurately transmitted from the SaMD terminal device 11 to the electronic medical record terminal device 21 without direct communication between the SaMD terminal device 11 and the electronic medical record terminal device 21. It is possible to accurately reflect the contents of the test result acquired by the medical service in the electronic medical record without necessity for a doctor or the like to manually input the contents of the test result displayed on the SaMD terminal device 11 to the electronic medical record terminal device 21 by using the operation part 211 or the like. Therefore, it is possible to reduce the labor of the doctor when registering the test result acquired by the medical service by SaMD in the electronic medical record.

After the approval of the patient and the doctor in Step S7 and Step S8, the reading in Step S12 is performed, and thus, only in a case where the patient and the doctor approve, the test result of the medical service by SaMD can be reflected in the electronic medical record. Thus, it is possible to prevent a situation in which the test result is reflected in the electronic medical record although the approvals are not acquired.

Furthermore, every time a medical service is used by the SaMD terminal device 11, the test result is registered in both the SaMD service providing server apparatus 12 and the electronic medical record server apparatus 22 (see Step S4 and Step S13 in FIG. 4). In this way, the usage history of the medical service is left on both the SaMD service providing system 10 side and the electronic medical record system 20 side, so that the medical service provider can charge an accurate usage fee according to the use result. In addition, since the usage history remains on the electronic medical record system 20 side, it is possible for the workers in clinical settings using the medical service to confirm that the usage fee charged by the medical service provider corresponds to the actual usage. Therefore, the medical service management system 100 according to Embodiment 1 can accurately manage the use of the medical service.

### Variation of Embodiment 1

The above-described Embodiment 1 may include the following variations.

### Variation 1

In the example of the operation of the medical service management system 100 according to Embodiment 1 illustrated in FIG. 4, the test result is displayed in a text format in Step S6, and after the approvals of the patient and the doctor are acquired in Step S7 and Step S8, the test result is displayed in a barcode format in Step S11. Thus, the patient and the doctor can perform approval after sufficiently understanding the contents of the test result. In addition, after the approvals of the patient and the doctor are acquired, the test result is transmitted from the SaMD terminal device 11 to the electronic medical record terminal device 21. Thus, it is possible to prevent a situation in which the test result is reflected in the electronic medical record although the approvals are not acquired.

In the present disclosure, the approvals of the patient and the doctor may not necessarily be performed before the display of the test result in a barcode format is permitted. That is, the SaMD terminal device 11 may acquire the approvals of the patient and the doctor while the test result is displayed in a text format and a barcode format. In this case, it is desired to adopt a configuration in which the test result displayed in a barcode format is not reflected in the electronic medical record before the approvals of the patient and the doctor. Specifically, after the approvals of the patient and the doctor are acquired, the SaMD service providing server apparatus 12 may register the test result in the electronic medical record managed by the electronic medical record server apparatus 22.

Furthermore, in some cases, the patient's approval may be omitted. For example, in a case where the degree of dementia of a patient is less than a predetermined level in a test result of a dementia test service performed on the patient, the SaMD terminal device 11 may permit display of the test result in a barcode format regardless of the patient's approval.

### Variation 2

The SaMD terminal device 11 may further have a function of printing the test result in a text format or a barcode format displayed on the display part 113. In this case, the SaMD terminal device 11 may have a printing function and print out at least one of the test result in a text format and/or the test result in a barcode format. Alternatively, the SaMD terminal device 11 may transmit the test result in a text format or a barcode format to an external printer or the like having a printing function, and cause the printer to perform printing. As a result, the test result can be stored in a paper format in clinical practice.

The SaMD terminal device 11 may further have a function of storing the display contents of the display part 113 as an image file. This case is preferred because information on the test result can be saved in the SaMD terminal device 11 that is generally used in clinical settings.

### Variation 3

In the medical service management system 100 according to the above-described Embodiment 1, the SaMD terminal device 11 transmits the acquired voice of the patient to the SaMD service providing server apparatus 12, and the SaMD service providing server apparatus 12 performs the test using the voice of the patient. The present disclosure is not limited thereto, and for example, the SaMD terminal device 11 may perform the test by itself without transmitting the voice of the patient to the SaMD service providing server apparatus 12. In this case, in the medical service management system, the SaMD terminal device may have a function of providing a medical service by SaMD. In this case, the medical service management system may not include the SaMD service providing server apparatus.

### Embodiment 2

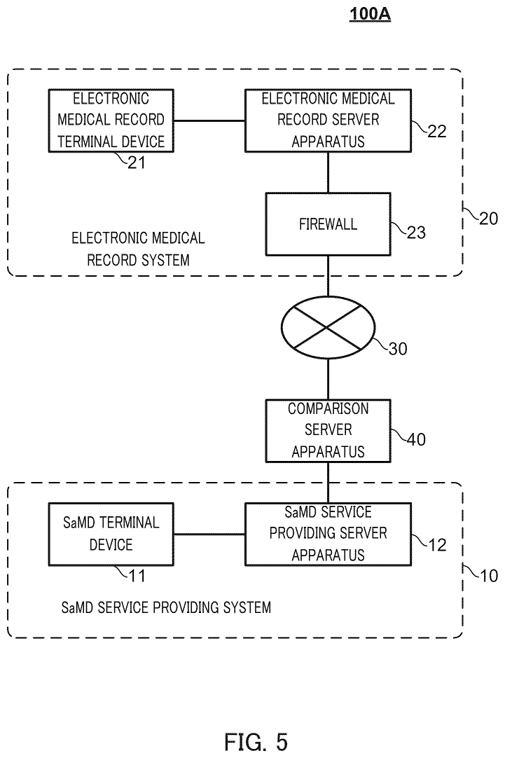

Embodiment 2 of the present disclosure is described below. Note that in Embodiment 2, the same components as those in Embodiment 1 are denoted by the same reference numerals as Embodiment 1, and detailed description of the components is omitted in some cases. FIG. 5 is a diagram illustrating an example of a configuration of the medical service management system 100A according to Embodiment 2 of the present disclosure. The medical service management system 100A according to Embodiment 2 is different from the medical service management system 100 described in Embodiment 1 in that the system includes a comparison server apparatus 40.

The comparison server apparatus 40 is located between the SaMD service providing system 10 and the electronic medical record system 20. Note that in the example illustrated in FIG. 5, the comparison server apparatus 40 is located between the network 30 and the SaMD service providing system 10, but the present disclosure is not limited thereto. The comparison server apparatus of the present disclosure may be located, for example, between the network and the electronic medical record system 20.

The comparison server apparatus 40 compares the usage history of the medical service managed on the side of the SaMD service providing system 10 with the usage history of the medical service managed on the side of the electronic medical record system 20, thereby managing accurate use results of the medical service. Examples of the comparison server apparatus 40 include a physical server composed of a stationary computer, and a cloud server configured by deploying one or a plurality of virtualized server functions in a cloud environment.

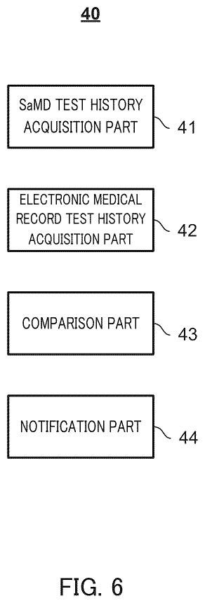

FIG. 6 is a block diagram illustrating an example of a functional configuration of the comparison server apparatus 40 according to Embodiment 2. As illustrated in FIG. 6, the comparison server apparatus 40 includes a SaMD test history acquisition part, an electronic medical record test history acquisition part 42, and a comparison part 43.

The SaMD test history acquisition part 41 acquires a usage history of a medical service managed on the SaMD service providing system 10 side. More specifically, the SaMD test history acquisition part 41 acquires the usage history from the SaMD terminal device 11 every time a medical service is used by the SaMD terminal device 11. Alternatively, the SaMD test history acquisition part 41 may collect and acquire, from the SaMD service providing server apparatus 12, a plurality of usage histories of the medical service for a certain period of time registered in the SaMD service providing server apparatus 12.

The electronic medical record test history acquisition part 42 acquires the usage histories of the medical service managed on the side of the electronic medical record system 20. More specifically, the electronic medical record test history acquisition part 42 acquires, from the electronic medical record server apparatus 22, the usage histories of the medical service registered in the electronic medical record managed by the electronic medical record server apparatus 22.

The comparison part 43 compares the usage histories of the medical service managed on the side of the SaMD service providing system 10 acquired by the SaMD test history acquisition part 41 with the usage histories of the medical service managed on the side of the electronic medical record system 20 acquired by the electronic medical record test history acquisition part 42. Thus, it is possible to confirm whether inconsistency occurs between the usage histories of the medical service managed on the side of the SaMD service providing system 10 and the side of the electronic medical record system 20, respectively.

In a case where inconsistency is found to have occurred as a result of the comparison by the comparison part 43, the notification part 44 notifies the SaMD service providing system 10 and the electronic medical record system 20 that inconsistency has occurred. This allows the medical professional in clinical settings and the business operator providing the medical service can promptly recognize the occurrence of the inconsistency. Thus, it is possible to match the usage history of the medical service managed on the side of the SaMD service providing system 10 with the usage history of the medical service managed on the side of the electronic medical record system 20.

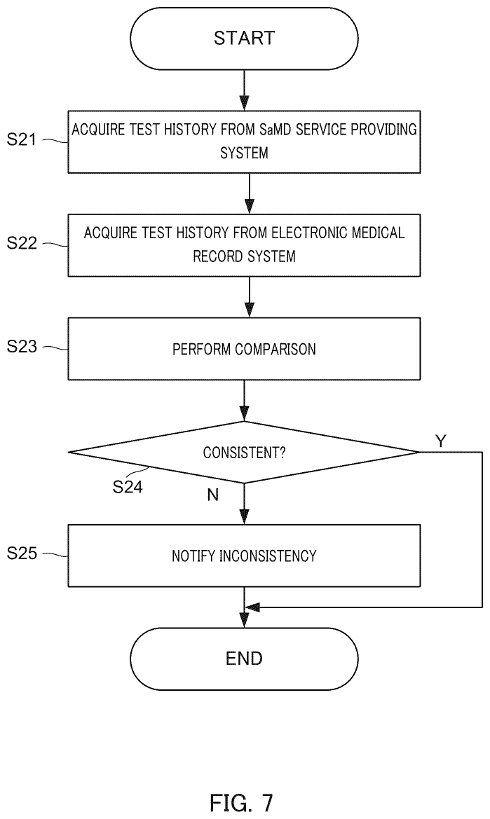

FIG. 7 is a flowchart illustrating an operation example at the time of comparison processing by the comparison server apparatus 40 in Embodiment 2. The comparison processing illustrated in FIG. 7 may be automatically executed by the comparison server apparatus 40, for example, at regular intervals.

In Step S21, the comparison server apparatus 40 acquires, from the SaMD service providing system 10, the usage history of the medical service managed on the side of the SaMD service providing system 10.

In Step S22, the comparison server apparatus 40 acquires, from the electronic medical record system 20, the usage history of the medical service managed on the side of the electronic medical record system 20.

In the example illustrated in FIG. 7, the processing of Step S21 is executed before the processing of Step S22. The present disclosure is not limited thereto, and the processing of Step S22 may be executed before Step S21. Alternatively, the processing of Step S21 and the processing of Step S22 may be performed substantially simultaneously.

In Step S23, the comparison server apparatus 40 compares the usage history acquired in Step S21 and managed by the SaMD service providing system 10 and the usage history acquired in Step S22 and managed by the electronic medical record system 20 together.

When the test histories are consistent with each other as a result of the comparison in Step S23 (Step S24:Y), the comparison server apparatus 40 ends the processing. When the test results are not consistent with each other (Step S24:N), the comparison server apparatus 40 advances the processing to Step S25.

In Step S25, the comparison server apparatus 40 notifies that the usage history managed on the side of the SaMD service providing system 10 and the usage history managed on the side of the electronic medical record system 20 are inconsistent with each other. The notification may be provided to the SaMD service providing system 10 and the electronic medical record system 20. Thus, for example, a medical professional who uses the SaMD service providing system 10 can grasp an inconsistency between the usage history managed by the SaMD service providing system 10 and the usage history managed by the electronic medical record system 20. Furthermore, for example, in the electronic medical record system 20, an administrator or the like of the electronic medical record can grasp the inconsistency between the usage history managed by the SaMD service providing system 10 and the usage history managed by the electronic medical record system 20. Thus, processing for resolving the inconsistency can be started on at least one of the SaMD service providing system 10 side and the electronic medical record system 20 side.

With the medical service management system 100A according to Embodiment 2, it is possible to compare the usage history managed on the SaMD service providing system 10 side and the usage history managed by the electronic medical record system 20 side. Then, in a case where the usage histories are inconsistent with each other, a notification can be provided. Thus, it is possible to prevent the business operator providing the medical service from charging a fee different from the use result when charging the usage fee for the medical service. Therefore, the medical service management system 100A according to Embodiment 1 can accurately manage the use of the medical service.

Although embodiments of the present invention have been described and illustrated in detail, the disclosed embodiments are made for purposes of illustration and example only and not limitation. The scope of the present invention should be interpreted by terms of the appended claims.

What is claimed is:

1. A medical service management system that manages use of a medical service by Software as a Medical Service (SaMD), wherein
the medical service management system causes:
a first terminal in which an application of the medical service is installed, to output display information indicating a test result by the medical service;
a second terminal linkable with an electronic medical record, to read the output display information; and
the test result to be registered in the electronic medical record based on a result of the reading.

2. The medical service management system according to claim 1, wherein
the medical service management system causes the first terminal to receive an approval from a doctor for the test result, and permits the first terminal to output the display information in response to receiving the approval from the doctor.

3. The medical service management system according to claim 2, wherein
the medical service management system causes the first terminal to receive an approval from a patient for the test result, and permits the first terminal to output the display information in response to receiving both the approval from the doctor and the approval from the patient.

4. The medical service management system according to claim 3, wherein
the medical service is a service for performing a diagnosis related to dementia based on a voice of the patient, and
the medical service management system receives the approval from the patient after the voice of the patient used for the diagnosis is reproduced.

5. The medical service management system according to claim 4, wherein
the medical service includes determination of a degree of the dementia in the patient, and
the medical service management system permits the first terminal to output the display information regardless of presence or absence of the approval from the patient, in a case where the degree of the dementia is less than a predetermined degree.

6. The medical service management system according to claim 1, wherein
the output of the display information by the first terminal is performed by causing a display included in the first terminal to display the display information.

7. The medical service management system according to claim 1, wherein
the output of the display information by the first terminal is performed by causing a printing apparatus linkable with the first terminal to print the display information.

8. The medical service management system according to claim 1, wherein
the medical service management system images the display information output by the first terminal, and causes the first terminal to store the display information as a test result image.

9. The medical service management system according to claim 1, wherein
the medical service management system manages the use of the medical service by comparing first history information and second history information together, the first history information being related to a usage history of the medical service generated by the first terminal, the second history information being generated based on the test result registered in the electronic medical record.

10. The medical service management system according to claim 9, wherein
the medical service management system causes:
an SaMD service providing server apparatus that provides the first terminal with a function of the medical service, to manage the first history information related to a plurality of the usage histories;
an electronic medical record server apparatus that provides the second terminal with a function of the electronic medical record, to manage the second history information based on a plurality of the test results; and
a comparison server apparatus independent of the SaMD service providing server apparatus and the electronic medical record server apparatus, to compare the first history information acquired from the SaMD service providing server apparatus and the second history information acquired from the electronic medical record server apparatus together.

11. The medical service management system according to claim 1, wherein
the medical service management system causes the first terminal to output the display information in a barcode format indicating the test result, and causes the second terminal to read the display information in the barcode format.
